(19) United States
(12) Patent Application Publication
(10) Pub. No.: US 2002/0068060 A1
(43) Pub. Date: Jun. 6, 2002

(54) Cytokine antagonists

(76) Inventors: Johannes Eduard Maria Antonius Debets, Rhoon (NL); John S. Abrams, Los Altos, CA (US); Robert A. Kastelein, Redwood City, CA (US); Anne O'Garra, Palo Alto, CA (US)

Correspondence Address: SCHERING-PLOUGH CORPORATION, PATENT DEPARTMENT (K-6-1, 1990), 2000 GALLOPING HILL ROAD, KENILWORTH, NJ, 07033-0530, US

(21) Appl. No.: 09/834,295

(22) Filed: Apr. 12, 2001

Related U.S. Application Data
(60) Provisional application No. 60/196,754, filed on Apr. 12, 2000.

(51) Int. Cl. A61K 39/395; A61K 39/40; A61K 39/42

(52) U.S. Cl. 424/145.1; 424/130.1; 424/139.1

(57) ABSTRACT

Antagonists of a cytokine signaling system have been found which exhibit favorable properties. In particular, antibody antagonists raised against the receptor are effective in blocking various signaling processes.

This application claims the benefit of priority from U.S. Provisional Patent Application No. 60/196,754, filed Apr. 12, 2000.

### FIELD OF THE INVENTION

The present invention relates generally to manipulation of mammalian physiology, particularly of cytokine mediated biology. More specifically, the invention relates to the modulation of IL-18 mediated physiology, including autoimmune or inflammatory conditions.

### BACKGROUND OF THE INVENTION

Recombinant DNA technology refers generally to the technique of integrating genetic information from a donor source into vectors for subsequent processing, such as through introduction into a host, whereby the transferred genetic information is copied and/or expressed in the new environment. Commonly, the genetic information exists in the form of complementary DNA (cDNA) derived from messenger RNA (mRNA) coding for a desired protein product. The carrier is frequently a plasmid having the capacity to incorporate cDNA for later replication in a host and, in some cases, actually to control expression of the cDNA and thereby direct synthesis of the encoded product in the host.

For some time, it has been known that the mammalian immune response is based on a series of complex cellular interactions, called the “immune network”. Recent research has provided new insights into the inner workings of this network. While it remains clear that much of the response does, in fact, revolve around the network-like interactions of lymphocytes, macrophages, granulocytes, and other cells, immunologists now generally hold the opinion that soluble proteins, known as lymphokines, cytokines, or monokines, play a critical role in controlling these cellular interactions. Thus, there is considerable interest in the isolation, characterization, and mechanisms of action of cell modulatory factors, an understanding of which will lead to significant advancements in the diagnosis and therapy of numerous medical abnormalities, e.g., immune system disorders. Some of these factors are hematopoietic growth and/or differentiation factors, e.g., stem cell factor (SCF) or IL-11. See, e.g., Mire-Sluis and Thorpe (1998) Cytokines Academic Press, San Diego; Thomson (ed. 1998) The Cytokine Handbook (3d ed.) Academic Press, San Diego; Metcalf and Nicola (1995) The Hematopoietic Colony Stimulating Factors Cambridge University Press; and Aggarwal and Gutterman (1991) Human Cytokines Blackwell.

Lymphokines apparently mediate cellular activities in a variety of ways. They have been shown to support the proliferation, growth, and differentiation of pluripotential hematopoietic stem cells into vast numbers of progenitors comprising diverse cellular lineages making up a complex immune system. Proper and balanced interactions between the cellular components are necessary for a healthy immune response, including development or maintenance. The different cellular lineages often respond in -a different manner when lymphokines are administered in conjunction with other agents.

Cell lineages especially important to the immune response include two classes of lymphocytes: B-cells, which can produce and secrete immunoglobulins (proteins with the capability of recognizing and binding to foreign matter to effect its removal), and T-cells of various subsets that secrete lymphokines and induce or suppress the B-cells and various other cells (including other T-cells) making up the immune network. These lymphocytes interact with many other cell types.

Inflammatory conditions are a significant medical problem. See, e.g., Gallin and Snyderman (eds. 1999) Inflammation: Basic Principles and Clinical Correlates; Ruffolo and Hollinger (eds. 1995) Inflammation: Mediators and Pathways; and Samter, et al. (eds.) Immunological Diseases vols. 1 and 2, Little, Brown and Co. The cytokines IL-12 and IL-18 have been implicated in various inflammatory conditions. See, e.g., Medline; Lebel-Binay, et al. (2000) Eur. Cytokine Netw. 11:15-26; Dinarello (1999) J. Allergy Clin. Immunol. 103:11-24; Okamura, et al. (1998) Curr. Op. Immunol. 10:259-64; and Okamura, et al. (1995) Nature 378:88-91. The IL-18 cytokine was early characterized as active in promoting proliferation and IFN-γ production by Th1 clones and lines, and NK cells in both mouse and human.

Okamura, et al. (1995) Nature 378:88-91; and Ushio, et al. (1996) J. Immunol. 156:4274-4279. Structural analysis and fold recognition suggested that IL-18 belongs to the IL-1 family. Bazan, et al. (1996) Nature 379:591(only). It has been shown to exhibit many other biological activities.

From the foregoing, it is evident that new means to treat inflammatory conditions are needed. In particular, the discovery and development of methodologies to enhance or potentiate the negative activities of known lymphokines would be highly advantageous. The present invention provides new methods for modulating inflammation.

### SUMMARY OF THE INVENTION

The present invention provides surprisingly advantageous methods of antagonizing IL-18 signaling. In particular, antagonizing the IL-18 cytokine may occur at various points in the signal pathway. In particular, the strategy of blocking signaling at the receptor provides a surprisingly better result than blocking at the ligand level.

The invention provides methods of antagonizing signaling mediated by IL-18 to a cell expressing the IL-18 receptor, said method comprising contacting said receptor with an antibody which binds to said receptor. Typically, the method is one where the signaling: is mediated by IRAK; induces IFN-γ induction by T or NK cells; or activates NF-κB.

In certain embodiments, the cell is an NK, Th1, or CD8+ cell, or in a host exhibiting symptoms of a chronic inflammatory or autoimmune response. In others, the host exhibits symptoms of rheumatoid arthritis, multiple sclerosis, or inflammatory bowel disease.

In other embodiments, the antibody: binds to the IL-1RD9 receptor subunit; competes with binding of antibody 28E3 to said receptor; or is the antibody 28E3.

The invention also provides such method in combination with antagonizing IL-12.

### DETAILED DESCRIPTION OF THE PREFERRED EMBODIMENTS

### Outline

I. General

A. IL-18

B. Receptor

II. Antagonists

A. Blocking ligand

B. Blocking receptor

III. Therapeutic compositions, methods

A. combination compositions

B. unit dose

C. administration

I. General

Interleukin-18 (interferon-γ-inducing factor; IGIF; IL-1γ; IL-18) was discovered in studies of IFN-γ production in a Propionobacterium acnes-induced model of toxic shock. Okamura, et al. (1995) Nature 378:88-91. This cytokine was subsequently characterized as active in promoting proliferation and IFN-γ production by Th1 clones and lines, and NK cells in both mouse and human. Okamura, et al. (1995) Nature 378:88-91; and Ushio, et al. (1996) J. Immunol. 156:4274-4279. Structural analysis and fold recognition suggested that IL-18 belongs to the IL-1 family. Bazan, et al. (1996) Nature 379:591(only).

Functionally, IL-18 potentiates IL-12 driven Th1 development. Robinson, et al. (1997) Immunity 7:571-581. In addition, IL-18 synergizes with IL-12 in inducing IFN-γ production from differentiating and committed Th1 cells from different strains of mice (Robinson, et al. (1997) Immunity 7:571-581) as well as inducing IFN-γ production by NK cells, in both mouse and humans, as well as T cell clones (Okamura, et al. (1995) Nature 378:88-91; and Ushio, et al. (1996) J. Immunol. 156:4274-4279). This suggested that both IL-12 and IL-18 were required for significant expression of the Th1 phenotype. See also, e.g., Medline; Lebel-Binay, et al. (2000) Eur. Cytokine Netw. 11:15-26; Dinarello (1999) J. Allergy Clin. Immunol. 103:11-24; Okamura, et al. (1998) Curr. Op. Immunol. 10:259-64; and Okamura, et al. (1995) Nature 378:88-91.

Unlike IL-12, IL-18 does not activate STAT4 in Th1 cells, but like other IL-1 family members, IL-18 signaling is via a receptor-associated kinase termed IRAK, which activates a cascade of kinases leading to activation of NF-κB. See Robinson, et al. (1997) Immunity 7:571-581; and Matsumoto, et al. (1997) Biochem. Biophys. Res. Commun. 234:454-457. That IL-18 signals the autophosphorylation of IRAK and the activation of NF-κb in Th1 cells, whereas IL-1α/β did not, first suggested that IL-18 binds and signals through a distinct receptor to the Type 1, IL-1R (R1/R3). Robinson, et al. (1997) Immunity 7:571-581; and O'Garra (1998) Immunity 8:275-283. This was further supported by the observations that IL-1α/β induced such signaling in Th2 but not Th1 cells, and furthermore by observations that IL-I8 does not activate this signaling pathway in Th2 cells. Robinson, et al. (1997) Immunity 15 7:571-581; and O'Garra (1998) Immunity 8:275-283. That the IRAK/MyD88/NF-kB pathway is required for both the actions of IL-1α/β and IL-18 (albeit on distinct cells) has now been confirmed, e.g., in IRAK (Kanakaraj, et al. (1999) J. Exp. Med. 189:1129-1138), and MyD88 (Adachi, et al. (1998) Immunity 9:143-150) gene-deleted mice, which lose both IL-1 and IL-18-mediated function. The requirement for Caspase-1 processing for both IL-1α/β and IL-18 action is also now evident. Fantuzzi, et al. (1998) Blood 91:2118-2125; and Ghayur, et al. (1997) Nature 386:619-623. Taken together, the observations that IL-12 and IL-18 synergize for Th1 responses (Robinson, et al. (1997) Immunity 7:571-581; even in the absence of TCR-triggering), and that each molecule utilizes a distinct signaling pathway, demonstrated the importance of this synergy between IL-12 and IL-18. This led to the hypothesis that signals through STAT4 and NF-κB are both implicated in host protection against intracellular pathogens, and in the induction of immunopathology. Robinson, et al. (1997) Immunity 7:571-581; and O'Garra (1998) Immunity 8:275-283. This theory was later supported by the findings that mice bearing targeted deletions of either the IL-12 or IL-18 genes produced a third of the levels of IFN-γ observed in wild-type controls in response to challenge with the mycobacterial antigen, BCG, in vivo. However, when mice were bred together so that they lacked both functional IL-12 and IL-18, the levels of IFN-γ produced by T cells upon rechallenge after BCG immunization were severely diminished even to the level produced by naïve T cells obtained from unimmunized mice (Takeda, et al. (1998) Immunity 8:383-390).

This suggested that, at least in response to this pathogen, IL-18 and IL-12 play distinct and non-redundant roles. This raised the possibility that intervention in the actions of either or both IL-12 and IL-18 might offer therapeutic potential, either in augmenting a protective Th1 responses as in chronic bacterial disease or anti-tumor. Osaki, et al. (1998) J. Immunol. 160:1742-1749; and Coughlin, et al. (1998) J. Clin. Invest. 101:1441-1452. Conversely their antagonists could function in diminishing damaging Th1 responses, e.g., in autoimmune diseases. Biological activities of IL-18 include inhibition of osteoclast proliferation, activation of NK cells and T cells, and augmentation of Th1 cell differentiation.

The specificity and binding characteristics of the IL-18 receptor (IL-18R) components have only been superficially explored. The IL-1RD5 (IL-1R DNAX number 5, see, e.g., PCT/US 98/20939 for DNAX numbered IL-1 receptor-like subunits; IL-1R related protein 1 (IL-1Rrpl; see U.S. Pat. No. 5,776,731) and IL-1RD9 (IL-1R DNAX number 9; also referred to as IL-1RAcPL (IL-1 accessory protein like; Immunex WO 99/37773; Born, et al. (1998) J. Biol. Chem. 273:29445-29450) confer responsiveness to IL-18 in a highly specific (no response to other IL-1 ligands) and unique manner (no functional pairing with other IL-1Rs and IL-1R-like molecules). See, e.g., PCT/US 98/20939 for DNAX numbered IL-1 receptor-like subunits. Co-transfection with both receptor components resulted in expression of both low-and high affinity binding sites for IL-18 (dissociation constants 11 and 0.4 nM, respectively). Blockage of the IL-18 signaling pathways would be expected to confer various therapeutic advantages in particular medical contexts.

The Th1/Th2 paradigm has proved useful for better understanding many clinical conditions. Th1- or Th2-dominated responses are not only responsible for distinct types of protection against infectious agents, but also appear to be involved in the pathogenesis of several immunopathological conditions, including some hypersensitivity states. For example, Th1 responses play a pathogenic role in organ-specific autoimmune disorders, whereas Th2-dominated responses against innocuous antigens (allergens) are responsible for atopic allergy. See, e.g., Romagnani (1997) The Th1/Th2 Paradigm in Disease Landes, Austin.

II. Antagonists

Blockage of the signaling pathway can be achieved by antagonists of the cytokine, e.g., antibodies to the ligand, antibodies to the receptor, antibodies to the individual receptor subunits, etc. Interference with the IL-18 ligand-receptor interaction has proven to be an effective strategy for the development of IL-18 antagonists. For instance, anti-IL-18 antibodies protect mice inoculated with Propionibacterium acnes and LPS from liver damage and inhibit progression of experimental acute encephalomyelitis in rats. See Okamura, et al. (1995) Nature 378:88-91; Wildbaum, et al. (1988) J. Immunol. 161:6368-6374; and Karin (1999). The soluble IL-18 binding protein inhibits bacterial-induced IFN-γ production. Novick, et al. (1999) Immunity 10:127-136; Yeda WO 99/09063. Moreover, anti-IL-1RD5 antibody reduces Th1 responses to LPS and Leishmania major. Xu, et al. (1998) J. Exp. Med. 188:1485-1492. Even though IL-1RD5 is a functional component of the IL-18R, its binding affinity for IL-18 is relatively low. Torigoe, et al. (1997) J. Biol. Chem. 272:25737-25742. However, both high- and low-affinity binding sites for IL-18 were observed on IL-12 pretreated T and B lymphocytes. Yoshimoto, et al. (1998) J. Immunol. 161:3400-3407. IL-1RD9, a candidate receptor to explain this discrepancy, has not been analyzed in great depth with respect to its role in ligand-receptor interaction or its expression on inflammatory effector cells and not at all with respect to its potential as a target to inhibit inflammatory pathologies.

There are various means to antagonize the signaling mediated by ligand. Two apparent means are to block the ligand with antibodies; a second is to block the receptor with antibodies. Various epitopes should exist on each which will block their interaction, e.g., causing steric hindrance blocking interaction. The correlation of ability to block signaling would not necessarily be expected to correlate with binding affinity to either ligand or receptors. The IL-1 receptor family members are hair-trigger receptors, e.g., each signaling receptor sends a very strong signal. As such, it would be expected that only a very few receptors on a cell would need to be activated to provide a full signal. And it would be expected that blocking at the receptor would be particularly difficult due to the requirement to block virtually every receptor on a cell. Such was observed in the IL-1α/IL-1β and IL-1RA system. Applicants have, in fact, compared high affinity antibodies against each of the ligand and the receptor, and find that the antibodies against the receptor work surprisingly well. In fact, similar amounts of antibody against the receptor provide a surprisingly better effectiveness in blocking signaling as compared against antibodies against the ligand.

The present invention provides for the use of an antibody or binding composition which specifically binds to an IL-18R, preferably mammalian, e.g., primate, human, cat, dog, rat, or mouse. Antibodies can be raised to various IL-18R proteins, including individual subunits or individual, polymorphic, allelic, strain, or species variants, and fragments thereof, both in their naturally occurring (full-length) forms or in their recombinant forms. Additionally, antibodies can be raised to IL-18R proteins in both their native (or active) forms or in their inactive, e.g., denatured, forms. Anti-idiotypic antibodies may also be used.

A number of immunogens may be selected to produce antibodies specifically reactive with IL-18R proteins. Recombinant protein is a preferred immunogen for the production of monoclonal or polyclonal antibodies. Naturally occurring protein, from appropriate sources, e.g., primate, rodent, etc., may also be used either in pure or impure form. Synthetic peptides, made using the human IL-1 8R protein sequences referenced, may also be used as an immunogen for the production of antibodies to IL-18R proteins. Recombinant protein can be expressed and purified in eukaryotic or prokaryotic cells as described, e.g., in Coligan, et al. (eds.) (1995 and periodic supplements) Current Protocols in Protein Science John Wiley & Sons, New York, N.Y.; and Ausubel, et al (eds.) (1987 and periodic supplements) Current Protocols in Molecular Biology, Greene/Wiley, New York, N.Y. Naturally folded or denatured material can be used, as appropriate, for producing antibodies. Either monoclonal or polyclonal antibodies may be generated, e.g., for subsequent use in immunoassays to measure the protein, or for immunopurification methods.

Methods of producing polyclonal antibodies are well known to those of skill in the art. Typically, an immunogen, preferably a purified protein, is mixed with an adjuvant and animals are immunized with the mixture. The animal's immune response to the immunogen preparation is monitored by taking test bleeds and determining the titer of reactivity to the IL-18R protein of interest. For example, when appropriately high titers of antibody to the immunogen are obtained, usually after repeated immunizations, blood is collected from the animal and antisera are prepared. Further fractionation of the antisera to enrich for antibodies reactive to the protein can be done if desired. See, e.g., Harlow and Lane; or Coligan. Immunization can also be performed through other methods, e.g., DNA vector immunization. See, e.g., Wang, et al. (1997) Virology 228:278-284.

Monoclonal antibodies may be obtained by various techniques familiar to researchers skilled in the art. Typically, spleen cells from an animal immunized with a desired antigen are immortalized, commonly by fusion with a myeloma cell. See, Kohler and Milstein (1976) Eur. J. Immunol. 6:511-519. Alternative methods of immortalization include transformation with Epstein Barr Virus, oncogenes, or retroviruses, or other methods known in the art. See, e.g., Doyle, et al. (eds.) (1994 and periodic supplements) Cell and Tissue Culture: Laboratory Procedures, John Wiley and Sons, New York, N.Y. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences which encode a monoclonal antibody or a binding fragment thereof by screening a DNA library from human B cells according, e.g., to the general protocol outlined by Huse, et al. (1989) Science 246:1275-1281.

Antibodies or binding compositions, including binding fragments and single chain versions, against predetermined fragments of IL-18R proteins can be raised by immunization of animals with conjugates of the fragments with carrier proteins as described above. Monoclonal antibodies are prepared from cells secreting the desired antibody. These antibodies can be screened for binding to normal or defective IL-18R protein, or screened for eosinophil depleting ability. These monoclonal antibodies will usually bind with at least a K_{D} of about 1 mM, more usually at least about 300 μM, typically at least about 10 μM, more typically at least about 30 μM, preferably at least about 10 μM, and more preferably at least about 3 μM or better.

In some instances, it is desirable to prepare monoclonal antibodies (mAbs) from various mammalian hosts, such as mice, rodents, primates, humans, etc. Description of techniques for preparing such monoclonal antibodies may be found in, e.g., Stites, et al. (eds.) Basic and Clinical Immunology (4th ed.) Lange Medical Publications, Los Altos, Calif., and references cited therein; Harlow and Lane (1988) Antibodies: A Laboratory Manual CSH Press; Goding (1986) Monoclonal Antibodies: Principles and Practice (2d ed.) Academic Press, New York, N.Y.; and particularly in Kohler and Milstein (1975) Nature 256:495-497, which discusses one method of generating monoclonal antibodies. Summarized briefly, this method involves injecting an animal with an immunogen. The animal is then sacrificed and cells taken from its spleen, which are then fused with myeloma cells. The result is a hybrid cell or “hybridoma” that is capable of reproducing in vitro. The population of hybridomas is then screened to isolate individual clones, each of which secrete a single antibody species to the immunogen. In this manner, the individual antibody species obtained are the products of immortalized and cloned single B cells from the immune animal generated in response to a specific site recognized on the immunogenic substance.

Other suitable techniques involve selection of libraries of antibodies in phage or similar vectors. See, e.g., Huse, et al. (1989) “Generation of a Large Combinatorial Library of the Immunoglobulin Repertoire in Phage Lambda,” Science 246:1275-1281; and Ward, et al. (1989) Nature 341:544-546. The polypeptides and antibodies of the present invention may be used with or without modification, including chimeric or humanized antibodies. Frequently, the polypeptides and antibodies will be labeled by joining, either covalently or non-covalently, a substance which provides for a detectable signal. A wide variety of labels and conjugation techniques are known and are reported extensively in both the scientific and patent literature. Suitable labels include radionuclides, enzymes, substrates, cofactors, inhibitors, fluorescent moieties, chemiluminescent moieties, magnetic particles, and the like. Patents teaching the use of such labels include U.S. Pat. Nos. 3,817,837; 3,850,752; 3,939,350; 3,996,345; 4,277,437; 4,275,149; and 4,366,241. Also, recombinant immunoglobulins may be produced, see, Cabilly, U.S. Pat. No. 4,816,567; and Queen, et al. (1989) Proc. Nat'l Acad. Sci. USA 86:10029-10033; or made in transgenic mice, see Mendez, et al. (1997) Nature Genetics 15:146-156; also see Abgenix and Medarex technologies.

Antibodies are merely one form of specific binding compositions. Other binding compositions, which will often have similar uses, include molecules that bind with specificity to IL-18R receptor, e.g., in a binding partner-binding partner fashion, an antibody-antigen interaction, or in a natural physiologically relevant protein-protein interaction, either covalent or non-covalent, e.g., proteins which specifically associate with IL-18R receptor protein. The molecule may be a polymer, or chemical reagent. A functional analog may be a protein with structural modifications, or may be a structurally unrelated molecule, e.g., which has a molecular shape which interacts with the appropriate binding determinants. Antibody binding compounds, including binding fragments, of this invention can have significant diagnostic or therapeutic value. They can be useful as non-neutralizing binding compounds and can be coupled to toxins or radionuclides so that when the binding compound binds to the antigen, a cell expressing it, e.g., on its surface, is killed. Further, these binding compounds can be conjugated to drugs or other therapeutic agents, either directly or indirectly by means of a linker, and may effect drug targeting.

III. Therapeutic compositions, methods

Antagonists to IL-18 mediated signaling have been shown to have significant therapeutic effects in various models. These include the in vitro assays of IL-18 induced stimulation of IFN-γ production by various cell types, both in mouse and human, e.g., Th1 cells and various cell lines, including transformed NKL (Robertson, et al. (1996) Exp. Hematol. 24:406-415), NK92, or cultured NK cells. In other models, the antagonists block clearance of Listeria monocytogenes infection and IFN-γ induction, and can affect the development of a memory response to the bacterial infection. In Legionella pneumophilus lung infection models, the antagonists can inhibit production of IFN-γ. In collagen induced arthritis models, the antagonist can effect positive results in decreasing arthritic score, paw swelling, and humoral response to collagen. In a mouse experimental autoimmune encephalomyelitis (EAE) model, which is a model for the human multiple sclerosis disease, the antagonist can delay onset of symptoms, and can slow down progression of the disease after onset. These collective models suggest that the regulation or development of Th1 type responses, infectious disease progression, autoimmune disease progression, and related degenerative diseases may be regulated by appropriate administration of the antagonist, temporally or otherwise.

The importance of regulating the Th1 vs. Th2 balance of the immune response are well described. See, e.g., Romagnani (1997) The Th1/Th2 Paradigm in Disease Landes, Austin; Powrie and Coffman (1993) Immunol. Today 14:270-274; and Sher and Coffman (1992) Ann. Rev. Immunol. 10:385-409. Similarly, the effects on response to infectious conditions are well known, and the therapeutic potential in its regulation is recognized.

Rheumatoid arthritis is a chronic and systemic autoimmune disease characterized by inflammation and progressive joint destruction, whose etiology is unclear and for which there is no known cure. Osteoarthritis shares many of these same problems. Proinflammatory cytokines have been implicated in disease pathogenesis. Additional studies have shown that a monoclonal antibody directed against the second chain of the IL-18R, e.g., IL-1RD9, will block anti-bacterial Th1 responses and MOG-induced EAE. Other results suggest that this monoclonal antibody can also inhibit onset of collagen-induced arthritis. The goal of any treatment is to reduce the inflammation and retard progression of joint and/or bone erosion. Significant medical need; despite currently available therapies, long term cost in dollars, and morbidity/increased mortality is significant. The treatment may be combined, e.g., with TNF inhibitors; Cox-2; ARAVA; Methotrexate; or other drugs for treatment of those symptoms.

Promising therapeutic effect has been suggested by studies showing inhibition of onset of experimental autoimmune encephalomyelitis (EAE) with anti-IL-18 antibodies (wildbaum, et al. (1998) J. Immunol. 161:6368-6374), and antibodies directed against the IL-18R (IL-1RD5 or IL-1Rrp) inhibited carrageenin-induced local inflammation (Xu, et al. (1998) J. Exp. Med. 188:1485-1492). EAE and multiple sclerosis typically are associated with the development of cellular immune responses within the central nervous system (CNS), characterized by the influx to the CNS of T lymphocytes and macrophages and, in some cases, a small number of granulocytes. Antibody production, thought to be involved in demyelination, also occurs in some experimental models of EAE and is a feature of MS, particularly those antibodies reacting with the cell-surface-expressed myelin protein, myelin oligodendrocyte glycoprotein (MOG). These responses are compared mechanistically to a delayed type hypersensitivity reaction, wherein the reaction is initiated by T lymphocytes of the T-helper 1 (Th1) type secreting the cytokines IL-2 and IFN-γ in abundance, and tissue damage is mediated by T cells as well as macrophages via production of various inflammatory and cytotoxic factors as well as via antibody/complement/ADCC-type reactions. At least in EAE, therapeutic regimens that target this broad immune reaction, including those directed towards development of the Th1 T cell, tend to be the most effective in disease amelioration or prevention. This includes blockade of cytokines such as IL-12, necessary for Th1 T cell development, tumor necrosis factor (TNF), involved in leukocyte recruitment, and TNF production/FasL expression, possibly involved in direct tissue damage within the CNS.

Multiple sclerosis (MS) is an inflammatory, demyelinating, and, to some extent, neuro-destructive disease of the CNS (300,000 cases in US, about a million world-wide; affects Caucasians), and a major cause of neurological disease in young adults. The disease course is variable, but the patient often deteriorates over time as a result of the dysfunction occurring from multiple attacks, or progressive disease. There is no effective therapy. The major aim would be to block the inflammatory process with minimum generalized immunosuppression, and minimal side effects (as compared to therapies presently used, which are not particularly effective).

The inflammatory bowel diseases of the gut encompassing Crohn's disease and ulcerative colitis are complex chronic diseases, seemingly Th1 mediated. The etiology and pathogenesis are poorly understood, but they afflict a significant number of patients with significant morbidity. These diseases are frequently relapsing diseases ultimately leading to destruction of mucosal tissue. Recent evidence suggests that a pathological activation of the mucosal immune system in response to antigens is a key factor in the pathogenesis of IBD. Furthermore, changes in cell migration and cytokine production appear to contribute to the perpetuation of IBD and the postoperative recurrence of Crohn's disease.

Collectively these studies suggest that antagonizing IL-18 or its receptor, with the appropriate entity may offer a therapeutic modality in diseases such as MS, IBD, or RA.

The antagonists of the present invention can be administered alone or in combination with another inhibitor of the same or accompanying pathway; or other compounds used for the treatment of symptoms, e.g., IL-12 antagonists, steroids such as glucocorticoids.

To prepare pharmaceutical or sterile compositions including the IL-18R antibody or binding composition thereof, the antibody or binding composition is admixed with a pharmaceutically acceptable carrier or excipient which is preferably inert. Preparation of such pharmaceutical compositions is known in the art, see, e.g., Remington's Pharmaceutical Sciences and U.S. Pharmacopeia: National Formulary, Mack Publishing Company, Easton, Pa. (1984).

Antibodies or binding compositions are normally administered parentally, preferably intravenously. Since such protein or peptide antagonists may be immunogenic they are preferably administered slowly, either by a conventional IV administration set or from a subcutaneous depot, e.g. as taught by Tomasi, et al, U.S. Pat. No. 4,732,863.

When administered parenterally the antibodies or fragments will be formulated in a unit dosage injectable form (solution, suspension, emulsion) in association with a pharmaceutically acceptable parenteral vehicle. Such vehicles are inherently nontoxic and nontherapeutic. The antagonist may be administered in aqueous vehicles such as water, saline, or buffered vehicles with or without various additives and/or diluting agents. Alternatively, a suspension, such as a zinc suspension, can be prepared to include the peptide. Such a suspension can be useful for subcutaneous (SQ) or intramuscular (IM) injection. The proportion of antagonist and additive can be varied over a broad range so long as both are present in effective amounts. The antibody is preferably formulated in purified form substantially free of aggregates, other proteins, endotoxins, and the like, at concentrations of about 5 to 30 mg/ml, preferably 10 to 20 mg/ml. Preferably, the endotoxin levels are less than 2.5 EU/ml. See, e.g., Avis, et al. (eds.)(1993) Pharmaceutical Dosage Forms: Parenteral Medications 2d ed., Dekker, N.Y.; Lieberman, et al. (eds. 1990) Pharmaceutical Dosage Forms: Tablets 2d ed., Dekker, N.Y.; Lieberman, et al. (eds. 1990) Pharmaceutical Dosage Forms: Disperse Systems Dekker, N.Y.; Fodor, et al. (1991) Science 251:767-773, Coligan (ed.) Current Protocols in Immunology; Hood, et al. Immunology Benjamin/Cummings; Paul (ed.) Fundamental Immunology; Academic Press; Parce, et al. (1989) Science 246:243-247; Owicki, et al. (1990) Proc. Nat'l Acad. Sci. USA 87:4007-4011; and Blundell and Johnson (1976) Protein Crvstallography, Academic Press, New York.

Selecting an administration regimen for an antagonist depends on several factors, including the serum or tissue turnover rate of the antagonist, the level of symptoms, the immunogenicity of the antagonist, and the accessibility of the target cells. Preferably, an administration regimen maximizes the amount of antagonist delivered to the patient consistent with an acceptable level of side effects. Accordingly, the amount of antagonist delivered depends in part on the particular antagonist and the severity of the condition being treated. Guidance in selecting appropriate doses is found in the literature on therapeutic uses of antibodies, e.g. Bach et al., chapter 22, in Ferrone, et al. (eds. 1985) Handbook of Monoclonal Antibodies Noges Publications, Park Ridge, N.J.; and Haber, et al. (eds.) (1977) Antibodies in Human Diagnosis and Therapy, Raven Press, New York, N.Y. (Russell, pgs. 303-357, and Smith, et al., pgs. 365-389).

Determination of the appropriate dose is made by the clinician, e.g., using parameters or factors known in the art to affect treatment or predicted to affect treatment. Generally, the dose begins with an amount somewhat less than the optimum dose and it is increased by small increments thereafter until the desired or optimum effect is achieved relative to any negative side effects. Important diagnostic measures include those of symptoms of the inflammation, e.g., level of inflammatory cytokines produced. Preferably, a IL-18R antibody or binding composition thereof that will be used is derived from the same species as the animal targeted for treatment, thereby minimizing a humoral response to the reagent.

The total weekly dose ranges for antibodies or fragments thereof, which specifically bind to IL-18R range generally from about 10 μg, more generally from about 100 μg, typically from about 500 μg, more typically from about 1000 μg, preferably from about 5 μg, and more preferably from about 10 mg per kilogram body weight. Generally the range will be less than 100 mg, preferably less than about 50 mg, and more preferably less than about 25 mg per kilogram body weight.

The weekly dose ranges for antagonists of IL-18 activity, e.g., antibody, binding fragments, or soluble receptors, range from about 1 μg, preferably at least about 5 μg, and more preferably at least about 10 μg per kilogram of body weight. Generally, the range will be less than about 1000 μg, preferably less than about 500 μg, and more preferably less than about 100 μg per kilogram of body weight. Dosages are on a schedule which effects the desired treatment and can be periodic over shorter or longer term. In general, ranges will be from at least about 10 μg to about 50 mg, preferably about 100 μg to about 10 mg per kilogram body weight.

The present invention also provides for administration of IL-18R antibodies or binding compositions in combination with known therapies, e.g., steroids, particularly glucocorticoids, which alleviate the symptoms associated with inflammation, or antibiotics or anti-infectives. Daily dosages for glucocorticoids will range from at least about 1 mg, generally at least about 2 mg, and preferably at least about 5 mg per day. Generally, the dosage will be less than about 100 mg, typically less than about 50 mg, preferably less than about 20 mg, and more preferably at least about 10 mg per day. In general, the ranges will be from at least about 1 mg to about 100 mg, preferably from about 2 mg to 50 mg per day. Suitable dose combinations with antibiotics, anti-infectives, or anti-inflammatories are also known.

The phrase “effective amount” means an amount sufficient to ameliorate a symptom or sign of the medical condition. Typical mammalian hosts will include mice, rats, cats, dogs, and primates, including humans. An effective amount for a particular patient may vary depending on factors such as the condition being treated, the overall health of the patient, the method route and dose of administration and the severity of side affects. When in combination, an effective amount is in ratio to a combination of components and the effect is not limited to individual components alone

An effective amount of antagonist will decrease the symptoms typically by at least about 10%; usually by at least about 20%; preferably at least about 30%; or more preferably at least about 50%. The present invention provides reagents which will find use in therapeutic applications as described elsewhere herein, e.g., in the general description for treating disorders associated with the indications described, e.g., rheumatoid arthritis, multiple sclerosis, inflammatory conditions, chronic or acute, etc. See, e.g., Dayer (1999) J. Clin. Invest. 104:1337-1339; Gracie, et al. (1999) J. Clin. Invest. 104:1393-1401; Berkow (ed.) The Merck Manual of Diagnosis and Therapy, Merck & Co., Rahway, N.J.; Thorn, et al. Harrison's Principles of Internal Medicine, McGraw-Hill, NY; Gilman, et al. (eds.) (1990) Goodman and Gilman's: The Pharmacological Bases of Therapeutics, 8th Ed., Pergamon Press; Remington's Pharmaceutical Sciences, 17th ed. (1990), Mack Publishing Co., Easton, Pa.; Langer (1990) Science 249:1527-1533; Merck Index, Merck & Co., Rahway, N.J.; and Physician's Desk Reference (PDR).

The broad scope of this invention is best understood with reference to the following examples, which are not intended to limit the inventions to the specific embodiments.

### EXAMPLES

I. General Methods

Some of the standard methods are described or referenced, e.g., in Maniatis, et al. (1982) Molecular Cloning, A Laboratory Manual, Cold Spring Harbor Laboratory, Cold Spring Harbor Press; Sambrook, et al. (1989) Molecular Cloning: A Laboratory Manual, (2d ed.), vols 1-3, CSH Press, NY; Ausubel, et al., Biology, Greene Publishing Associates, Brooklyn, N.Y.; or Ausubel, et al. (1987 and Supplements) Current Protocols in Molecular Biology, Greene/Wiley, New York. Methods for protein purification include such methods as ammonium sulfate precipitation, column chromatography, electrophoresis, centrifugation, crystallization, and others. See, e.g., Ausubel, et al. (1987 and periodic supplements); Deutscher (1990) “Guide to Protein Purification” in Meth. Enzymol., vol. 182, and other volumes in this series; and manufacturer's literature on use of protein purification products, e.g., Pharmacia, Piscataway, N.J., or Bio-Rad, Richmond, Calif. Combination with recombinant techniques allow fusion to appropriate segments, e.g., to a FLAG sequence or an equivalent which can be fused via a protease-removable sequence. See, e.g., Hochuli (1989) Chemische Industrie 12:69-70; Hochuli (1990) “Purification of Recombinant Proteins with Metal Chelate Absorbent” in Setlow (ed.) Genetic Engineering. Principle and Methods 12:87-98, Plenum Press, N.Y.; and Crowe, et al. (1992) QIAexpress: The High Level Expression & Protein Purification System QIAGEN, Inc., Chatsworth, Calif.

Computer sequence analysis is performed, e.g., using available software programs, including those from the GCG (U. Wisconsin) and GenBank sources, Public sequence databases were also used, e.g., from GenBank and others.

Many techniques applicable to IL-4 and IL-10 may be applied to IL-18, as described, e.g., in U.S. Pat. No. 5,017,691 (IL-4), U.S. Ser. No. 07/453,951 (IL-10), and U.S. Ser. No. 08/110,683 (IL-10 receptor). Furthermore, aspects of the various receptor subunits have been described, and antagonists may be used as described, e.g., with IL-10 receptor in U.S. Pat. No. 5,789,192. See also, PCT/US 98/20939 for DNAX numbered IL-1 receptor-like subunits; IL-1R related protein 1 (IL-1Rrpl; see U.S. Pat. No. 5,776,731) and IL-1RD9 (IL-1R DNAX number 9; also referred to as IL-1RAcPL (IL-1 accessory protein like; Immunex WO 99/37773; Born, et al. (1998) J. Biol. Chem. 273:29445-29450). A soluble IL-18 binding protein has also been described. See Novick, et al. (1999) Immunity 10:127-136; and Yeda WO 99/09063. Each of these is incorporated herein by reference for all purposes

II. Characterization of IL-18R and monoclonal antibodies

Searching the expressed sequence tag database with the intracellular portion of mouse IL-1R3 revealed an EST (Genbank accession no. AA203986) with sequence homology to the IL-1 receptor family. A probe was made on the basis of this sequence and used to clone full length mouse IL-1RD9 (IL-1 receptor DNAX designation 9) cDNA from a spleen cDNA library. Using the mouse sequence, two human EST's were discovered in a data base that encoded the human homologue of mouse IL-1RD9. Full length human IL-1RD9 cDNA was isolated from a human spleen cDNA library.

IL-1RD9 was identified as a component of the receptor for IL-18 by combining IL-1RD9 with other IL-1 receptors and a NF-κB reporter construct transiently transfected into Jurkat cells. Transient expression and combination of IL-1RD9 and IL-1RD5 in Jurkat cells in the presence of IL-18 led to the induction of NF-κB, as measured by the activity of luciferase, a reporter gene under control of NF-κB.

Scatchard analysis was performed of IL-18 binding to Jurkat cells expressing IL-1RD5 and IL-1RD9. Binding experiments on Jurkat cells transiently transfected with IL-1RD5 and IL-1RD9 using iodinated IL-18 indicate the presence of two classes of receptor for this ligand with different affinities. Scatchard analysis reveals high and low affinity binding sites with Kd's of 370 pM (540 sites) and 11 nM (4360 sites), respectively. The low affinity receptor presumably reflects the interaction of IL-18 with the primary binding receptor IL-1RD5, whereas the high affinity receptor signaling complex consists of IL-18, IL-1RD5, and IL-1RD9.

Both the IL-1RD9 and IL-1RD5 chains of the IL-18R are expressed on Th1 cells and down regulated on Th2 cells. Analysis of IL-18R mRNA expression on a panel of cells showed that both the IL-1RD5 and IL-1RD9 are highly expressed on Th1 cells and down-regulated to low or undetectable levels on Th2 cells. This could be seen irrespective of the state of activation of these cells. Naïve T cells expressed very low levels of the IL-1RD5, but low to undetectable levels of the IL-1RD9 chain, and both chains were not detected on pre-T cells. Most tissues expressed low to undetectable levels of both chains of the IL-18R, although the IL-1RD5 chain was detected at low levels in lung tissue. Endothelial cells, mast cells, B cells, dendritic cells, and macrophages expressed little to undetectable levels of either IL-1RD5 or IL-1RD9, except for a highly activated preparation of the macrophage cell line J774, which expressed low levels of the IL-1RD9.

A series of monoclonal antibodies were raised and identified, against the IL-1RD9 chain of the IL-18R. These monoclonal antibodies were then tested for their ability to inhibit IL-18 induced IFN-γ production by Th1 cells (in synergy with IL-12; and LPS-induced IFN-γ production from mouse spleen cells.

III. Measuring IL-18 activity; neutralizing monoclonal antibodies

RPMI 1640 (JR Scientific Inc., Woodland Calif.) supplemented with 10% fetal calf serum (JR Scientific Inc.), 2-Mercaptoethanol (0.05 mM, Sigma, St. Louis, Mich.), L-glutamine (2 mM), penicillin (100 U/ml), streptomycin (100 mg/ml), HEPES buffer (10 mM), and sodium pyruvate (1 mM) was used as culture medium (cRPMI). For dendritic cells isolations, RPMI 1640 Dutch modification (Gibco, Life Technologies Ltd, Irvine, Scotland) was used with glutamine, fetal calf serum, penicillin, and streptomycin as described above.

Recombinant mouse cytokines were as follows; IL-18 (IGIF) was produced by expression in E. coli (DNAX); recombinant mouse IL-12 was obtained from Pharmingen, San Diego, Calif.; Th1 clone HDK1 were used in experiments at least ten days after their last antigen stimulation, and after culture in medium with IL-2 alone. Cells were stimulated in 96 well plates at 5×10⁴ cells/well for 48 h with either medium in the presence or absence of IL-18 (2 ng/ml) and/or IL-12 (0.2 ng/ml) added either separately or in combination. Supernatants were collected at 48 h and assayed for IFN-γ. Monoclonal antibodies were added as supernatants or purified at the indicated concentrations.

Antibodies were raised against human IL-18 in collaboration with Dr. J. Wijdeness of Diaclone (France). A series of hybridoma supernatants that initially reacted positive in an IL-18 binding assay and subsequently, purified antibodies, were tested for neutralization of IL-18 bioactivity on NKL cells. NKL cells were cultured in the presence of IL-2 or of IL-2+IL-18 (10 ng/ml) in the absence or presence of 1:5 dilutions of 50 μg/ml purified anti-IL-18 monoclonal antibodies. Supernatants were harvested after 48 h and IFN-γ production was measured by ELISA. See, e.g., Abrams (1995) Curr. Protocols in Immunol. 13:6.1. Many monoclonal antibodies were able to block the activity of IL-18 with monoclonal antibodies 2A9 and 5D11 showing the strongest neutralizing activity. This assay is suitable for screening hybridoma supernatants of antibodies that interfere with components of IL-18 dependent receptor-ligand interactions.

IFN-γ was detected using a two-site sandwich ELISA. Other cytokines may be assayed by similar methods.

Antibodies were selected for the most potent neutralizing monoclonal antibody in hybridoma supernatants containing antibodies directed against the IL-1RD9 chain of the IL-18R. The assay consisted of Th1 cells stimulated to produce IFN-γ in response to known amounts of added IL-18 and IL-12, which synergize for this effect. Of a large number of supernatants tested, a clone designated 28E3 was selected as the most potent in neutralizing this activity. This particular hybridoma clone has been deposited with the ATCC under deposit no: PTA-1690. This monoclonal antibody's effects were also the most potent neutralizers of LPS-induced IFN-γ production by mouse spleen cells. Production of this monoclonal antibody was then scaled up and the antibody purified to homogeneity. When compared to available anti-IL-18 monoclonal antibodies directed against the IL-18 ligand itself, the anti-IL-18R (IL-1RD9) antibody 28E3 showed a surprisingly greater efficacy in neutralizing the activity of IL-18 to induce IFN-γ from Th1 cells, in synergy with IL-12. This was an impressive ten-fold greater efficacy in vitro.

Alternatively, the inhibition of mouse IL-18 signaling through IL-1RD9 by 28E3 was measured by transiently transfecting Jurkat cells with IL-1RD5, IL-1RD9, and the luciferase reporter construct. Cells were subsequently incubated with either IL-18 alone or in combination with excess 28E3 antibody. The amount of luciferase activity produced was strongly reduced in the presence of 28E3, indicating blocking of IL-1RD9 by this antibody. Importantly the antibody demonstrated no agonist activity. The anti-IL-18R monoclonal antibody showed greater antagonist activity than soluble receptors.

Similar antibodies can be selected using the methods and screens described, and even more effective clones should be found by optimization processes.

All citations herein are incorporated herein by reference to the same extent as if each individual publication or patent application was specifically and individually indicated to be incorporated by reference.

Many modifications and variations of this invention can be made without departing from its spirit and scope, as will be apparent to those skilled in the art. The specific embodiments described herein are offered by way of example only, and the invention is to be limited by the terms of the appended claims, along with the full scope of equivalents to which such claims are entitled; and the invention is not to be limited by the specific embodiments that have been presented herein by way of example.

What is claimed is:

1. A method of antagonizing signaling mediated by IL-18 to a cell expressing the IL-18 receptor, said method comprising contacting said receptor with an antibody which binds to said receptor.

2. The method of claim 1, wherein said signaling:
a) is mediated by IRAK; or
b) induces IFN-γ induction by T or NK cells.

3. The method of claim 2, wherein said signaling is mediated by IRAK.

4. The method of claim 2, wherein said signaling induces IFN-γ induction by T or NK cells.

5. The method of claim 4, wherein said signaling induces IFN-γ induction by T cells.

6. The method of claim 1, wherein said signaling activates NF-κB.

7. The method of claim 1, wherein said cell is in a host exhibiting symptoms of a chronic inflammatory or autoimmune response.

8. The method of claim 7, wherein said cell is:
a) an NK, Th1, or CD8+ cell; or
b) in a host exhibiting symptoms of a chronic inflammatory response.

9. The method of claim 7, wherein said cell is in a host exhibiting symptoms of an autoimmune response.

10. The method of claim 1, wherein said cell is in a host exhibiting symptoms of rheumatoid arthritis, multiple sclerosis, or inflammatory bowel disease.

11. The method of claim 10, wherein said cell is in a host exhibiting symptoms of rheumatoid arthritis.

12. The method of claim 10, wherein said cell is in a host exhibiting symptoms of multiple sclerosis.

13. The method of claim 10, wherein said cell is in a host exhibiting symptoms of inflammatory bowel disease.

14. The method of claim 1, wherein said antibody binds to the IL-1RD9 subunit.

15. The method of claim 1, wherein said antibody competes with binding of antibody 28E3 to said receptor.

16. The method of claim 2, wherein said antibody competes with binding of antibody 28E3 to said receptor.

17. The method of claim 7, wherein said antibody competes with binding of antibody 28E3 to said receptor.

18. The method of claim 10, wherein said antibody competes with binding of antibody 28E3 to said receptor.

19. The method of claim 1, wherein said antibody is antibody 28E3.

20. The method of claim 1, in combination with antagonizing IL-12.

21. The method of claim 7, in combination with antagonizing IL-12.
